(19) United States
(12) Patent Application Publication
(10) Pub. No.: US 2023/0212530 A1
(43) Pub. Date: Jul. 6, 2023

(54) VIRUS PURIFICATION METHOD USING APATITE COLUMN

(71) Applicant: HOYA TECHNOSURGICAL CORPORATION, Tokyo (JP)

(72) Inventor: Yae KUROSAWA, Tokyo (JP)

(73) Assignee: HOYA TECHNOSURGICAL CORPORATION, Tokyo (JP)

(21) Appl. No.: 18/008,526

(22) PCT Filed: Jun. 16, 2020
(86) PCT No.: PCT/JP2020/023627

§ 371 Date: Dec. 6, 2022

(51) Int. Cl. C12N 7/00

(52) U.S. Cl. CPC C12N 7/00; C12N 2770/32623; C12N 2770/32651

(57) ABSTRACT

[Problems] The present invention provides a purification method capable of more effectively removing the contaminants with physical characteristics similar to the virus from the composition containing the virus, while preventing the denaturation of the virus, than conventional methods.

[Means to solve problems] A purification method for removing a contaminant from a composition containing a virus particle and the contaminant, the purification method comprising: a preparation step of preparing the composition containing a virus particle and a contaminant; a first adsorption step of adsorbing the virus particle on a first adsorbent composed of a calcium phosphate compound by contacting the composition with the first adsorbent; a first elution step of eluting the virus particle from the first adsorbent to obtain a first eluate; a second adsorption step of adsorbing the virus particle on a second adsorbent composed of a calcium phosphate compound by contacting the first eluate with the second adsorbent; and a second elution step of eluting the virus particle from the second adsorbent to obtain a second eluate; one of the first elution step and the second elution step comprising pH gradient elution, and the other comprising salt concentration gradient elution.

### FIELD OF THE INVENTION

The present invention relates to a virus purification method using a ceramic apatite column.

More specifically, the present invention relates to a sequential two-step chromatographic purification of an infectious poliovirus using a ceramic fluoroapatite column and a ceramic hydroxyapatite column.

The background and summary of the purification method of the present invention are as follows. Infectious virus purification techniques are important for vaccine development and gene therapy applications. However, the standardized one-step purification technique using ceramic hydroxyapatite (CHAp) has proven unsuitable for poliovirus. Therefore, we designed a sequential two-step chromatographic method for purification of the infectious Sabin type 2 vaccine strain of poliovirus from the cell culture supernatant. In the first step, we removed protein contaminants from the Sabin type 2 virus fraction by pH gradient elution on a ceramic fluoroapatite column. In the second step, we removed double-stranded DNA derived from host cells by diluting the virus fraction, directly loading it on a CHAp column, and purifying it using a phosphate gradient with 1 M sodium chloride. This process achieved removal rates of 99.95% or more and 99.99% or more for proteins and double-stranded DNA, respectively, and was highly reproducible and scalable. Furthermore, it is likely that it will be applicable to other virus species.

### BACKGROUND OF THE INVENTION

The development of safer vaccines demands a reduction in their side effects. Hence, regulatory authorities such as the Food and Drug Administration (FDA) require both developers and manufacturers to improve the production and purification processes. Various procedures have been developed for vaccine purification, including ultracentrifugation, liquid chromatography (LC), electrophoresis, precipitation, and molecular sieving. In particular, ultracentrifugation using cesium chloride, sucrose, or iodixanol gradients has been used to process several viral preparations for pharmaceutical use. However, these procedures decrease the infectivity of some viruses and is unable to separate particulate contaminants with physical characteristics similar to the virus particles, such as host cell DNA, from the virus fraction. In addition, density-gradient separation is time-consuming and has low scalability and economic efficiency.

There is a current trend toward using liquid chromatography for the purification of viral preparations because it is easy to use from the early capture stage to the final purification phase, and offers a straightforward scale-up, unlike traditional centrifugal procedures. Furthermore, it has recently been used for large-scale purification (>10¹³ virus particles) using several approaches, including ion exchange, size exclusion, hydrophobic interaction, immobilized metal affinity, and hydroxyapatite chromatography.

For biosafety reasons, hydroxyapatite is commonly used in biomaterial engineering and regenerative medicine as well as for the purification of pharmaceutical products. Recently, our team standardized the hydroxyapatite chromatography procedure and successfully purified dengue virus type 2 and Japanese encephalitis virus from the cell culture supernatants of virus-infected C6/36 cells and mouse brain homogenate, respectively, using one-step hydroxyapatite liquid chromatography. Scanning electron microscopy analysis confirmed that the virus particles were bound to and released from the surface of the hydroxyapatite via the chromatographic processes, clearly indicating the phosphate-dependent adsorption/desorption mechanism of hydroxyapatite. Therefore, we considered that apatite-based materials would be suitable for the purification of vaccines for diseases and vectors for gene therapies, increasing their effectiveness and reducing their cost.

Two kinds of polio vaccines are currently available: the Salk and Sabin vaccines. The Salk vaccine is an injectable vaccine of formalin-inactivated virulent poliovirus strains, whereas the Sabin vaccine is an oral vaccine that uses live attenuated virus strains. The Sabin oral vaccine causes occasional incidences of vaccine-associated paralytic poliomyelitis by a circulating vaccine-derived poliovirus. Hence, the development of a noninfectious method of vaccination has become a priority. Consequently, an injectable Sabin vaccine has recently been developed from safer strains, and it is now possible for small companies to manufacture this vaccine with a smaller investment in facilities. This is because the Sabin strain is less transmissible than the other wild-type strains and does not require a high level of biosafety. Polioviruses belong to acid-stable Picornaviridae and retain their infectivity at pH 3 and lower. The spherical virus particles do not contain a lipid envelope, have a diameter of approximately 30 nm, and are composed of four structural proteins: VP1, VP2, VP3, and VP4.

As a purification method specialized for poliovirus, for example, Patent Reference 1 discloses a method using a cation exchange chromatography or a size exclusion chromatography. However, this technique likely causes the denaturation of viruses by using the ion exchange chromatography. Furthermore, this purification method has a problem such that samples are diluted by using the size exclusion chromatography, thereby necessitating concentrating the diluted samples, resulting in a low production efficiency.

### PATENT REFERENCE

Patent Reference 1: WO 2016/012445

### OBJECT OF THE INVENTION

An object of the present invention is thus to provide a purification method capable of effectively removing the contaminants with physical characteristics similar to the virus from the composition containing the virus, while preventing the denaturation of the virus.

### SUMMARY OF THE INVENTION

This object can be achieved by the present invention described below.

[A]

A purification method for removing a contaminant from a composition containing a virus particle and the contaminant, the purification method comprising:

a preparation step of preparing the composition containing a virus particle and a contaminant;

a first adsorption step of adsorbing the virus particle on a first adsorbent composed of a calcium phosphate compound by contacting the composition with the first adsorbent;

a first elution step of eluting the virus particle from the first adsorbent to obtain a first eluate;

a second adsorption step of adsorbing the virus particle on a second adsorbent composed of a calcium phosphate compound by contacting the first eluate with the second adsorbent; and

a second elution step of eluting the virus particle from the second adsorbent to obtain a second eluate;

one of the first elution step and the second elution step comprising pH gradient elution, and the other comprising salt concentration gradient elution.

[B]

The purification method described in [A], wherein the pH gradient elution comprises a step of changing a pH value of a solution supplied to the first adsorbent from a first pH value to a second pH value,

the first pH value is acidic, and

the second pH value is neutral or alkaline.

[C]

The purification method described in [B], wherein the first pH value is 6.0 or lower, and the second pH value is 7.5 or higher.

[D]

The purification method described in [A], wherein the salt concentration gradient elution comprises a step of changing a salt concentration of a solution supplied to the second adsorbent from a first concentration to a second concentration,

the first concentration is 10 mM or less, and

the second concentration is 100 mM or more.

[E]

The purification method described in [A], wherein the first elution step comprises the pH gradient elution, the first adsorbent comprising fluoroapatite,

the second elution step comprises the salt concentration gradient elution, the second adsorbent comprising hydroxyapatite, and

the first elution step is conducted before the second elution step.

[F]

The purification method described in [A] or [B], wherein the virus particle is a poliovirus particle.

[G]

A method for producing a virus particle, the method comprising a step of removing a contaminant from the composition by the purification method described in [A] or [B].

### Effects of the Invention

The purification method of the present invention can more effectively remove the contaminants with physical characteristics similar to the virus from the composition containing the virus, while preventing the denaturation of the virus, than conventional methods.

### BRIEF DESCRIPTION OF THE DRAWINGS

FIG. 1 shows a standardized two-step purification procedure.

FIG. 2A shows the purification of the Sabin type 2 vaccine by pH gradient elution on a ceramic fluoroapatite (CFAp) column.

FIG. 2B shows an analysis of the fraction using SDS-PAGE.

FIG. 3A shows a chromatogram of Sabin type 2 virus-containing cell culture supernatant purified in the presence of 1.0 M NaCl.

FIG. 3B shows a chromatogram of Sabin type 2 virus-containing cell culture supernatant purified in the presence of 1.5 M NaCl.

FIG. 4A shows the purification (step 1) of the Sabin type 2 vaccine by pH gradient elution.

FIG. 4B shows the purification (step 2) of the Sabin type 2 vaccine by salt concentration gradient elution.

FIG. 4C shows an analysis of the fraction obtained by two-step purification using SDS-PAGE.

FIG. 5 shows a tandem column system to which the purification method of the embodiment of the present invention is applied.

FIG. 6A shows a chromatogram of Sabin type 2 virus-containing cell culture supernatant separated on a ceramic hydroxyapatite (CHAp) column at pH 6.4.

FIG. 6B shows a chromatogram of Sabin type 2 virus-containing cell culture supernatant separated on a ceramic hydroxyapatite (CHAp) column at pH 7.2.

FIG. 6C shows a chromatogram of Sabin type 2 virus-containing cell culture supernatant separated on a ceramic hydroxyapatite (CHAp) column at pH 8.2.

FIG. 7A shows a chromatogram of dengue virus type 1-containing cell culture supernatant separated on a ceramic hydroxyapatite (CHAp) column at pH 6.4.

FIG. 7B shows a chromatogram of dengue virus type 1-containing cell culture supernatant separated on a ceramic hydroxyapatite (CHAp) column at pH 7.2.

FIG. 7C shows a chromatogram of dengue virus type 1-containing cell culture supernatant separated on a ceramic hydroxyapatite (CHAp) column at pH 8.2.

FIG. 8A shows a chromatogram of influenza virus NYMC X-181 containing cell culture supernatant separated on a ceramic hydroxyapatite (CHAp) column at pH 6.5.

FIG. 8B shows a chromatogram of influenza virus NYMC X-181 containing cell culture supernatant separated on a ceramic hydroxyapatite (CHAp) column at pH 6.8.

FIG. 8C shows a chromatogram of influenza virus NYMC X-181 containing cell culture supernatant separated on a ceramic hydroxyapatite (CHAp) column at pH 7.5.

FIG. 9A shows a chromatogram of influenza virus NYMC X-181 containing cell culture supernatant in the absence of NaCl (0 M).

FIG. 9B shows a chromatogram of influenza virus NYMC X-181 containing cell culture supernatant in the presence of NaCl (0.14 M).

FIG. 9C shows a chromatogram of influenza virus NYMC X-181 containing cell culture supernatant in the presence of NaCl (0.5 M).

FIG. 9D shows a chromatogram of influenza virus NYMC X-181 containing cell culture supernatant in the presence of NaCl (1 M).

### DESCRIPTION OF THE PREFERRED EMBODIMENTS

The preferred embodiments of the present invention will be described below.

Adsorbents for use in the pH gradient elution are preferably usable even under acidic conditions. Being usable even under acidic conditions means that even when the pH value of the solution loaded on the column is lower than 7.0, the adsorbents are substantially neither dissolved nor decomposed, exhibiting their separation functions.

It is preferable that the adsorbents for use in the pH gradient elution are mainly composed of fluoroapatite represented by formula: Ca₁₀(PO₄)₆(F)₂. The fluoroapatite preferably has a spherical particle shape. As the fluoroapatite of a spherical particle shape, for example, CFT (ceramic fluoroapatite) available from Bio-Rad Laboratories, Inc. may be used.

The pH gradient means that the pH value is changed from the first pH value to the second pH value, the first pH value being acidic, the second pH value being neutral or alkaline.

From the viewpoint of increasing the retention volumes of viruses, the first pH is preferably 3.0 to 7.0, more preferably 3.5 to 6.5, further preferably 4.0 to 6.0, and most preferably 4.5 to 5.5.

the second pH is preferably 7.4 to 9.0, more preferably 7.6 to 8.8, further preferably 7.8 to 8.6, and most preferably 8.0 to 8.4.

It is preferable that the adsorbents for use in the salt concentration gradient elution are mainly composed of hydroxyapatite represented by formula: Ca₁₀(PO₄)₆(OH)₂. The hydroxyapatite preferably has a spherical particle shape. As the hydroxyapatite of a spherical particle shape, for example, CHT TYPE1 and CHT TYPE2 available from Bio-Rad Laboratories, Inc. may be used.

A salt used for eluting viruses by the salt concentration gradient is preferably a phosphate buffering agent. That is, an eluent for eluting viruses is preferably a phosphate buffer solution. The phosphate buffer solution includes, for example, an aqueous solution containing phosphate such as sodium phosphate, potassium phosphate, lithium phosphate and ammonium phosphate. In addition, the buffer solution may be a 2-morpholinoethanesulfonic acid (MES) buffer solution or a sulfate such as sodium sulfate.

The salt concentration gradient is preferably to increase the salt concentration in the eluent from the first concentration to the second concentration.

The first concentration is preferably 1 mM to 50 mM.

The second concentration is preferably 80 mM to 270 mM.

When the phosphate buffer solution is used as the eluent, the phosphate buffer solution may further contain other salt than phosphate. The other salt may be a salt of alkali metal or alkali earth metal. The other salt may include NaCl, KCl, NH₄Cl and MgCl₂, and is preferably NaCl.

The other salt concentration may be 0.1 M or more and 4.0 M or less, and is preferably 0.2 M or more and 2.5 M or less, more preferably 0.5 M or more and 2.0 M or less, further preferably 0.75 M or more and 1.75 M or less, and most preferably 1.0 M or more and 1.5 M or less. The phosphate buffer solution containing the other salt of such concentrations can achieve the separation of the viruses with higher purity.

From the viewpoint of preventing hydroxyapatite from being dissolved, the pH of the eluent used in the salt concentration gradient elution is preferably 7.0 to 9.0, more preferably 7.05 to 8.0, and further preferably 7.1 to 7.5.

The removal rate of the double-stranded DNA (dsDNA) by the purification method of the present invention is preferably 97% by mass or more per 100% by mass of the total amount of dsDNA included in the sample before conducting these steps. The removal rate is more preferably 98% by mass or more, further preferably 99% by mass or more, and most preferably 99.9% by mass or more.

The flow rate of the buffer in the elution processes in the steps 1 and 2 is preferably about 0.1 mL/min or more and 10 mL/min or less, and more preferably 0.2 mL/min or more and 5 mL/min or less, from the viewpoint of reducing the time required for separation operation and reliably separating the target.

Either of the pH gradient elution and the salt concentration gradient elution may be carried out first. By carrying out both, both protein contaminants and DNA contaminants can be removed. It is preferable that the pH gradient elution is carried out first, and then the salt concentration gradient elution is carried out. The reason is as follows. The DNA contaminants in the sample are excessively adsorbed on the adsorbents in the presence of the other salt such as chloride sodium for use in the salt concentration gradient elution. Therefore, by removing the DNA contaminants in the sample via the pH gradient elution before the sample is used for the salt concentration gradient elution, the amount of the DNA contaminants, which are adsorbed on the adsorbents and hardly separated from virus in the salt concentration gradient elution, is reduced, resulting in higher purity of the virus obtained. Otherwise, the eluate obtained in the preceding elution may be diluted, and then supplied for the next elution.

The purification method of the present invention is preferably used for a compound having a positively or negatively charged portion. The purification method of the present invention is preferably directed to a positively or negatively charged protein, more preferably directed to RNA viruses such as poliovirus, calicivirus and influenza virus, and DNA viruses such as adenovirus, further preferably directed to viruses that are not substantially deactivated under acidic conditions such as poliovirus.

Herein, “being not substantially deactivated” means that the infectivity of the virus-containing sample is not substantially reduced under conditions such as acidic conditions. In the embodiment, the “substantially no deactivated” infectivity of the sample is preferably 70% or more, more preferably 80% or more, further preferably 90% or more, and most preferably 95% or more, of its original infectivity.

Herein, that a composition A is “mainly composed of” a compound B means that the amount of the compound B is 50% by mass or more per 100% by mass of the total amount of the composition A. In the embodiment, this ratio is preferably 70% by mass or more, more preferably 80% by mass or more, further preferably 90% by mass or more, and most preferably 95% by mass or more.

### Example

Here, we report on the design and experimental validation of a sequential liquid chromatographic procedure using ceramic fluoroapatite (CFAp) and CHAp for purification of the Sabin type 2 strain of poliovirus.

Materials and Methods

Preparation of Sabin Type 2 Virus-Containing Cell Culture Supernatant

Vero cells (the American Type Culture Collection, Manassas, Va., USA) were cultured in minimum essential medium (MEM; Thermo Fisher Scientific Inc., Waltham, Mass., USA) containing 10% fetal bovine serum (FBS; Thermo Fisher Scientific Inc.) and L-glutamine (2 mM; Thermo Fisher Scientific Inc.) in 225-cm² flasks (Sumitomo Bakelite Co., Ltd., Tokyo, Japan) at 37° C. in 5% CO₂ for 3 days. The medium was then changed to MEM containing 2% FBS and 2 mM L-glutamine, and the cells were grown for a further day. After changing the medium to MEM (63 mL) without FBS, Sabin type 2 virus was inoculated onto the cell monolayer at MOI 0.01 and cultured at 37° C. for 2 days. The cell culture supernatant was then collected, passed through a 0.45-μm filter (polyethersulfone membrane; Thermo Fisher Scientific Inc.) to remove cell debris, and stored without any stabilizer at −80° C. until use. The virus was highly stable under this condition and showed no change in its TCID₅₀ value during the 4-month storage period (data not shown).

CHAp and CFAp Columns

CHT Ceramic Hydroxyapatite, Type II (CHAp; 40-μm particle size) and CFT Ceramic Fluoroapatite, Type II (CFAp; 40-μm particle size) were purchased from Bio-Rad Laboratories Inc. (Hercules, Calif., USA). Both are ceramic-type materials with strict specifications. The particles were packed into empty stainless steel columns (4.6 mm i.d.×35 mm; Sugiyama Shoji Co., Ltd., Kanagawa, Japan) in-house using a dry method.

Chromatographic Procedures

Chromatography was performed using a BioLogic DuoFlow™ system (Bio-Rad Laboratories Inc.) with a 10- or 20-mL sample loop at a flow rate of 1.0 mL/min. The samples were loaded onto a CHAp/CFAp column and eluted using a linear concentration gradient of sodium phosphate buffer (NaPB) ranging from 10 mM to 600 mM. The resulting eluate was monitored for ultraviolet (UV) absorbance at 260 and 280 nm and for conductivity. The collected fractions were kept at 4° C. and were immediately used for the evaluations described below. Before use, the running buffers were filtered (0.22 μm). The fraction collector was placed in a biological safety cabinet, and the attached tubes were autoclaved. In addition, the interiors of the pumps, columns, and lines were sterilized with ethanol for disinfection (Amakasu Chemical Industries, Tokyo, Japan), washed with autoclaved ultrapure water followed by 600 mM NaPB, and equilibrated with 10 mM NaPB before use. After the experiments, the column and system were sterilized with 0.5M NaOH for 10 column volumes and washed with autoclaved ultrapure water to remove the alkaline solution.

Measurement of Infectivity of the Virus-Containing Fractions

The Sabin type 2 virus titer was obtained by measuring the median tissue culture infectious dose (TCID₅₀) using a confluent monolayer of Vero cells in 96-well microplates. To prepare the confluent monolayer, Vero cells (100 μL, 1×10⁵ cells/mL) were cultured in MEM containing 10% FBS at 37° C. for 1 day. Each fraction obtained by liquid chromatographic separation was 10-fold serially diluted with MEM containing 10% FBS, and the resulting diluents (50 μL) were inoculated into each well (n=3) and cultured for 1 week. Each well of the microplates was then examined under a light microscope CKX31 (Olympus Corporation, Tokyo, Japan) to determine whether cytopathic effects had occurred. Titers were calculated using the Reed-Muench method.

Other Evaluations

The concentrations of double-stranded DNA (dsDNA) and proteins were determined using the Quant-iT™ PicoGreen dsDNA Assay Kit (Thermo Fisher Scientific Inc.) and the Micro BCA™ Protein Assay Kit (Thermo Fisher Scientific Inc.) according to the manufacturer's instructions. For sodium dodecyl sulfate-polyacrylamide gel electrophoresis (SDS-PAGE), the liquid chromatographic fractions were concentrated using a ultrafiltration (Ultracel YM-10; Millipore Corporation, Billerica, Mass., USA) and applied to 15% polyacrylamide gel (c-PAGEL; ATTO Corporation, Tokyo, Japan). Each protein band was visualized by silver staining, and the molecular weight was calculated by the Gel Doc™ EZ Imager (Bio-Rad Laboratories Inc.).

Results

Two-Step Procedure for the Purification of Sabin Type 2 Virus

We initially used one-step CHAp chromatography to purify live attenuated Sabin type 2 virus. However, this proved unsuccessful because both protein and DNA contaminants could not be separated from the virus fraction using this method. Therefore, we attempted to introduce separation by CFAp chromatography as the first step before conducting CHAp chromatography. The strategy of this two-step chromatographic procedure is outlined in FIG. 1. In Step 1, virus particles were directly separated from the cell culture supernatant containing a complex protein mixture of infected cells by CFAp chromatography using pH gradient elution. In Step 2, the resulting virus fraction was separated by CHAp chromatography using NaPB concentration gradient elution with a high concentration of sodium chloride (NaCl) to remove contaminated dsDNA from the fraction. We optimized each step as described below to separate the virus.

FIG. 1 shows a standardized two-step purification procedure.

CFAp (ceramic fluoroapatite)

CHAp (ceramic hydroxyapatite)

NaPB (sodium phosphate buffer)

Step 1: pH gradient elution of Sabin type 2 virus from a CFAp column

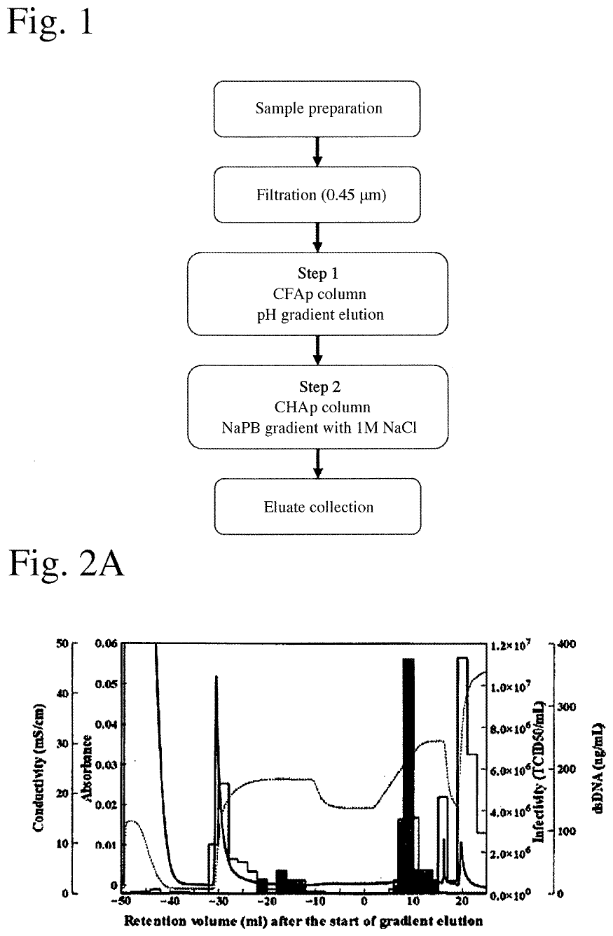

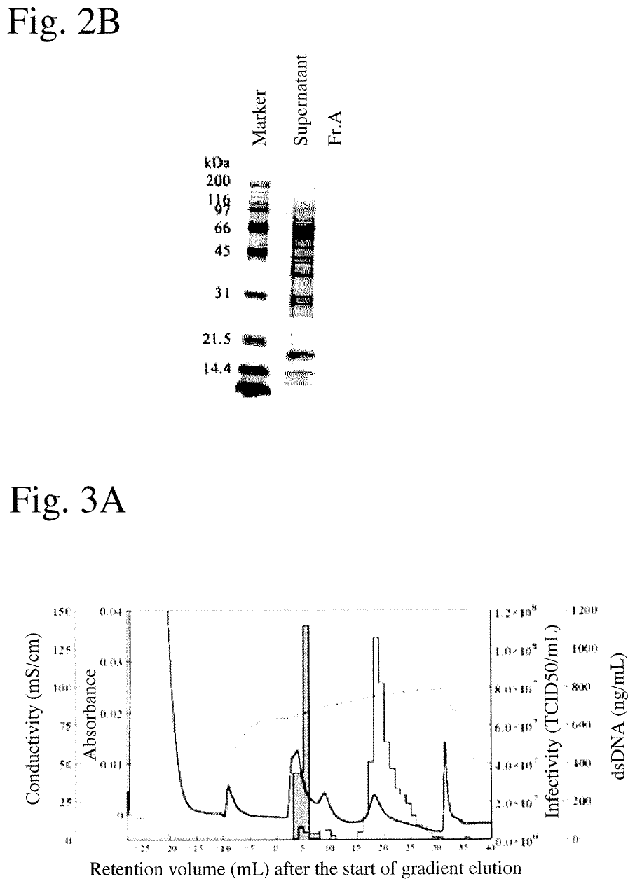

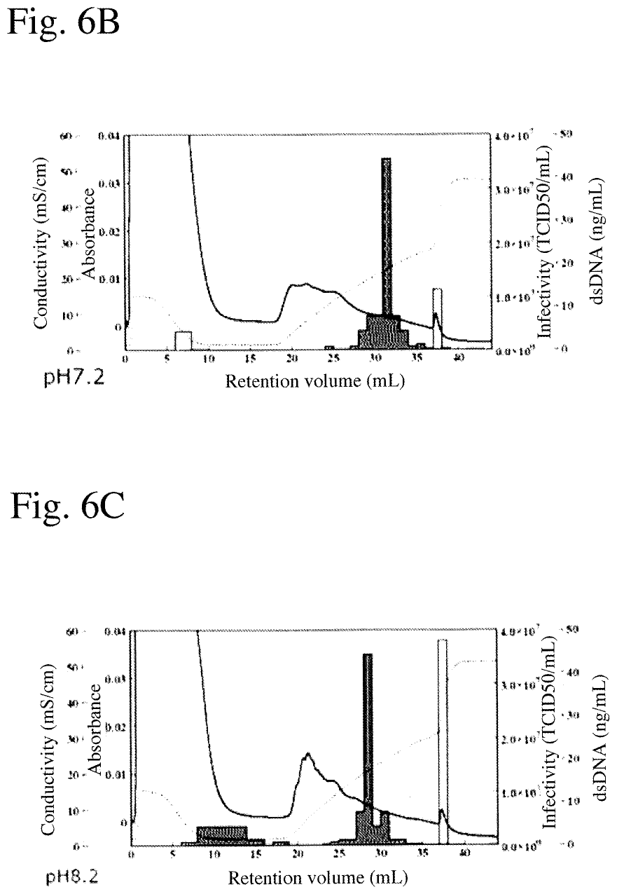

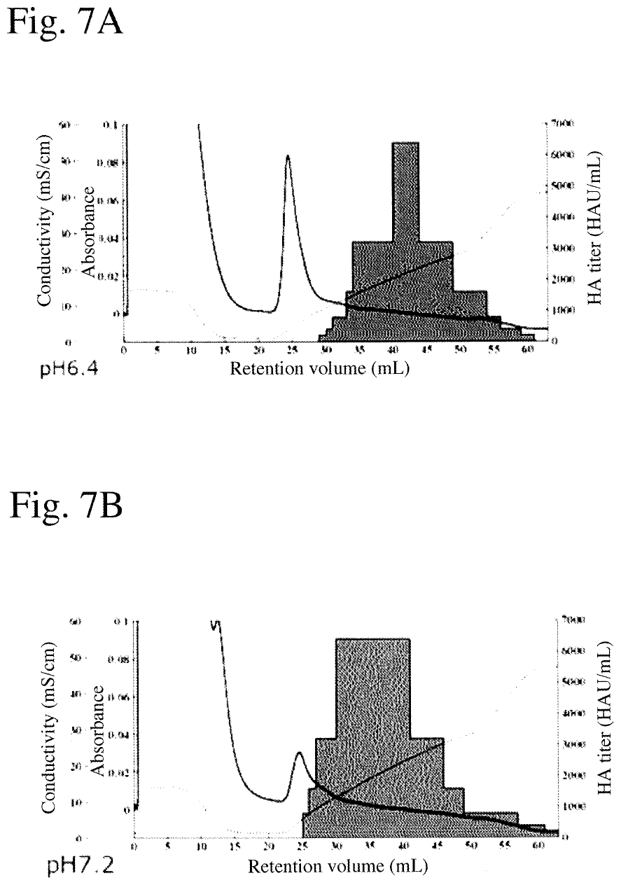

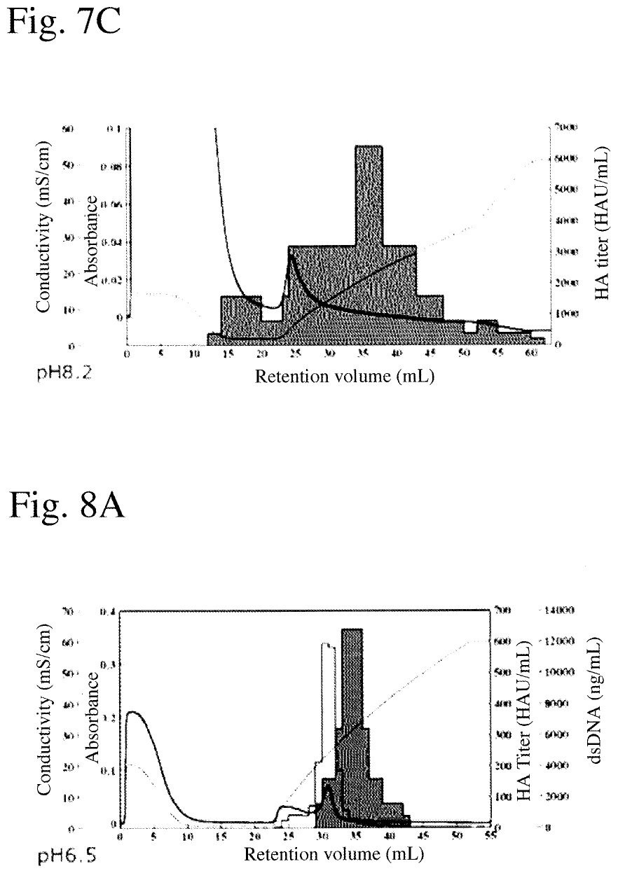

We separated three viruses [poliovirus Sabin type 2; dengue virus type 1 (Hawaii) (Department of Virology, Institute of Tropical Medicine, Nagasaki University, Japan); and influenza virus NYMC X-181 (National Institute for Biological Standards and Control, Hertfordshire, UK)] on a CHAp column by elution with a similar concentration gradient of NaPB at different pH values (FIGS. 6A to 8C). We found that the retention volumes of these viruses increased as the pH of the running buffer was lowered, regardless of the physicochemical properties of the virus. However, at pH 6.4, the Sabin type 2 virus fraction still contained protein contaminants (FIG. 6A), whereas the dengue virus fraction appeared to have been separated from the protein fraction (FIG. 7A). Therefore, we then attempted to apply CFAp chromatography, which shows similar separation to CHAp but allows a further reduction in the pH of the running buffer due to its greater acid tolerance (applicable pH range, 5 to 14) compared with CHAp (applicable pH range, 6.5 to 14). We separated Sabin type 2 virus from the cell culture supernatant on a CFAp column using a linear pH gradient from pH 5.0 to 8.2 in 300 mM NaPB because use of a pH gradient reduces the contact time between the low pH and the virus (FIG. 2A). The mean retention volume of the virus was found to be 9.6±0.44 mL (11 independent measurements, mean±standard deviation), indicating that the separation was highly reproducible. The resulting fraction (“Fr. A” indicated in FIG. 2A) showed a mean recovery rate of 94.1%±42.2% (11 independent measurements) in TCID₅₀ and a mean protein removal rate of 91.87% (two independent measurements). Analysis of the fraction using SDS-PAGE showed that there were three bands at approximately 37, 32, and 30 kDa (FIG. 2B), which agreed with the previously reported separation patterns of the capsid proteins VP1, VP2, and VP3 of Sabin type 2 virus, indicating that the virus had been separated from the protein contaminants Nevertheless, the fraction still contained dsDNA (mean removal rate, 88.15%; two independent measurements) derived from the host cells that needed to be removed.

The contents of the experiments in FIG. 2 will be described below.

FIG. 2 shows the purification of Sabin type 2 virus by pH gradient elution on a ceramic fluoroapatite (CFAp) column.

(A) Chromatogram obtained under the following conditions:

Column, CFAp;

sample (volume), cell culture supernatant containing Sabin type 2 virus (5 mL);

column wash, 10 mM sodium phosphate buffer (NaPB; pH 6.4, 10 mL) and 300 mM NaPB (pH 6.4, 20 mL);

equilibration, 300 mM NaPB (pH 5, 15 mL);

elution, linear pH gradient at 300 mM NaPB from pH 5 to pH 8.2 for 10 mL; and

wash after separation, 300 mM NaPB (pH 8.2, 5 mL) and 600 mM NaPB (pH 8.2, 10 mL);

blue line, ultraviolet (UV) absorbance at 280 nm;

black broken line, conductivity;

red line, infectivity in median tissue culture infectious dose (TCID₅₀); and

purple line, double-stranded DNA (dsDNA) contents.

“Fr. A” was pooled for the evaluation.

(B) Sodium dodecyl sulfate-polyacrylamide gel electrophoresis (SDS-PAGE) analysis

The cell culture supernatant and pooled Fr. A were concentrated 10-fold and 30-fold, respectively, by ultrafiltration using a molecular weight cutoff of 10,000. The molecular weights of the marker proteins are given in kDa.

Step 2: Separation of Sabin Type 2 Virus from Double-Stranded DNAs by CHAp

Effect of NaCl Addition to the Mobile Phase

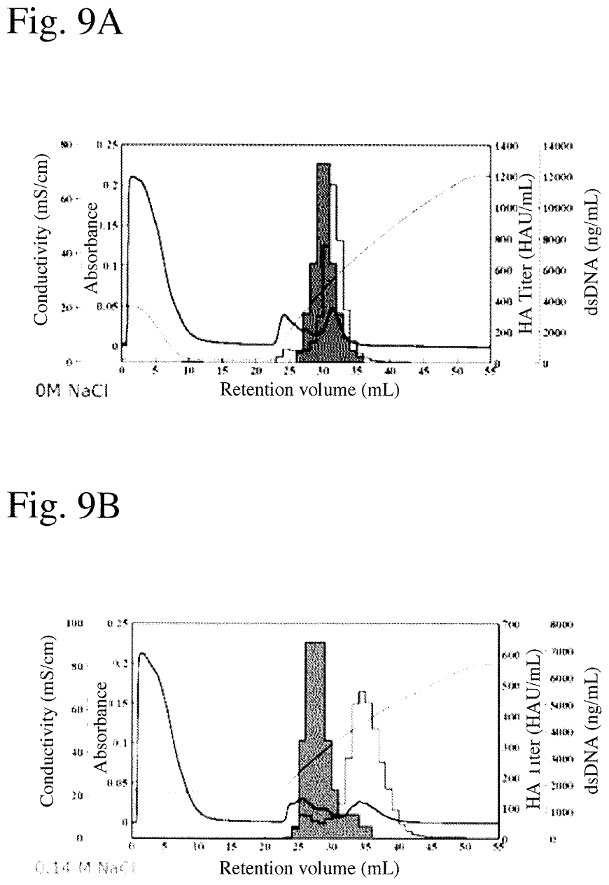

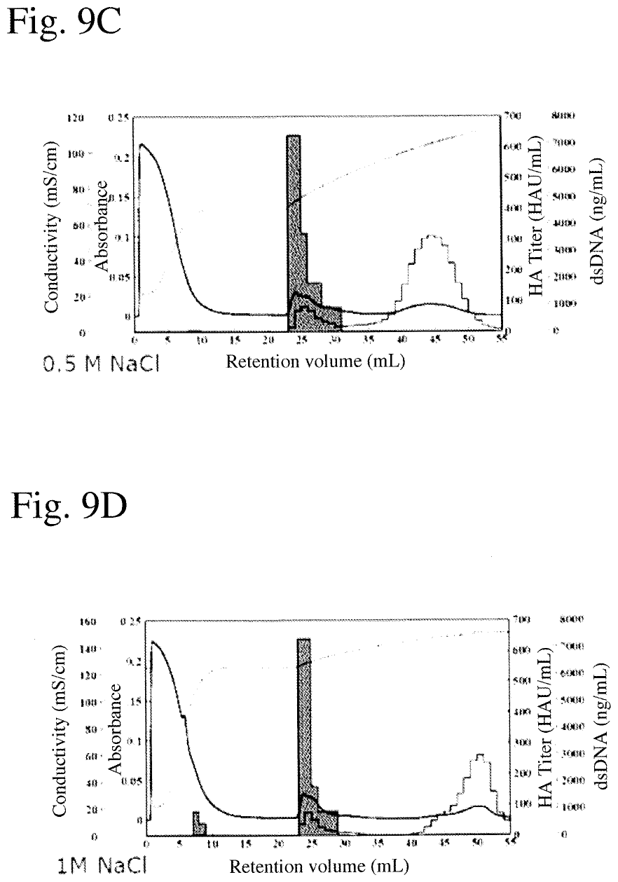

We next examined whether a high concentration of NaCl in the mobile phase of Step 2 would allow double-stranded DNA to be removed from the virus fraction based on the knowledge that excess sodium ions on the phosphate site of hydroxyapatite decrease the electrostatic repulsion between the phosphate site and DNA, causing an increase in the relative affinity of the calcium site for DNA. To evaluate this, we separated two viruses (influenza virus NYMC X-181 and feline calicivirus) in the presence of 0 to 1.5M NaCl. We found that the retention volume of double-stranded DNA increased with increasing NaCl concentrations in the running buffer, whereas the retention volume of the virus particles decreased (FIG. 9). Thus, a high concentration of NaCl in the mobile phase proved effective in removing double-stranded DNA from the virus fraction. We used 1 and 1.5 M NaCl in the mobile phase to separate Sabin type 2 virus from double-stranded DNA in the cell culture supernatant (FIG. 3). An increased retention of double-stranded DNA compared with the virus particle was obtained in both separations, indicating that the addition of 1 M NaCl was sufficient to remove double-stranded DNA from the virus peak (removal rate, 97.32%). Furthermore, this separation was highly reproducible, with a virus retention volume after the start of concentration gradient elution of 5.0±0.5 mL (three independent measurements).

The contents of the experiments in FIG. 3 will be described below.

FIG. 3 shows the chromatograms of Sabin type 2 virus-containing cell culture supernatant in the presence of NaCl. Separation was carried out in the presence of (A) 1 M NaCl and (B) 1.5 M NaCl under the following conditions:

Column, ceramic hydroxyapatite (CHAp);

sample (volume), cell culture supernatant containing Sabin type 2 virus (5 mL);

buffer pH, 7.2;

column wash, 10 mM sodium phosphate buffer (NaPB; 9 mL);

equilibration, 10 mM NaPB with NaCl (14 mL);

elution, linear concentration gradient from 10 mM to 600 mM NaPB with NaCl for 30 mL; and

wash after separation, 600 mM NaPB (10 mL).

Lines are the same as in FIG. 2.

TCID₅₀, median tissue culture infectious dose.

Sequential Two-Step Chromatographic Purification of Sabin Type 2 Virus

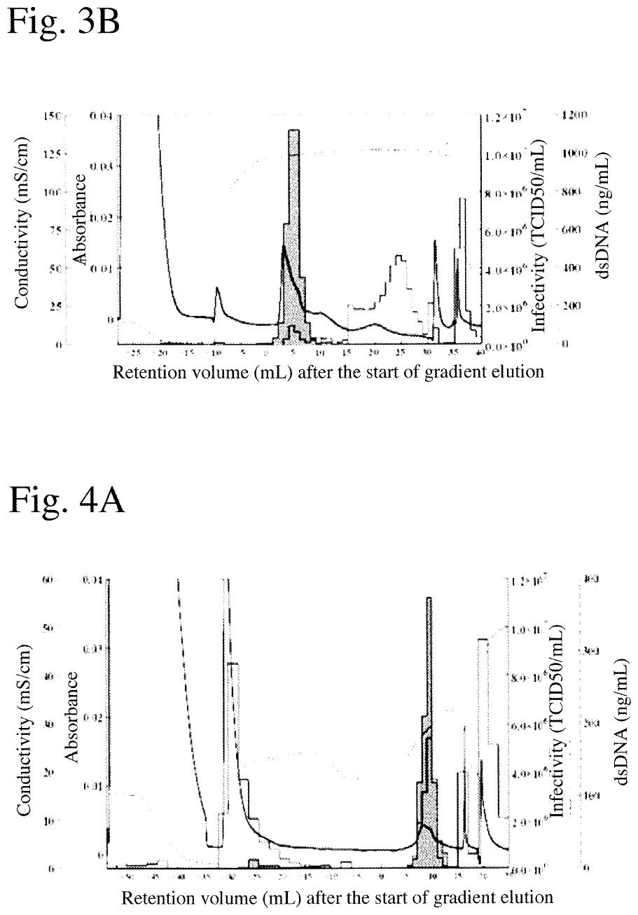

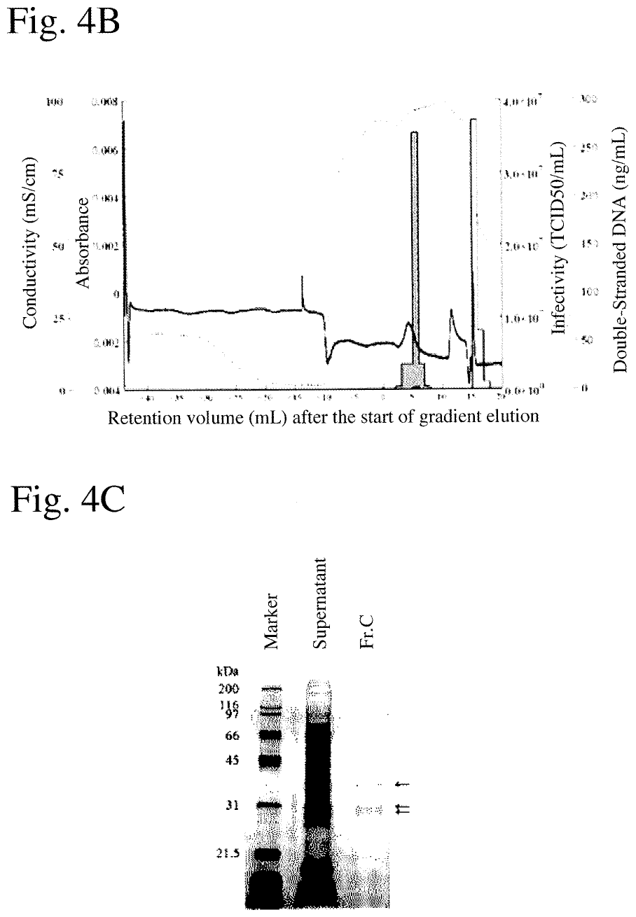

We performed the two-step sequential procedure (Steps 1 and 2 described above) to isolate Sabin type 2 virus from the cell culture supernatant. A representative case is shown in FIG. 4.

The virus particles were separated by pH gradient elution on a CFAp column (FIG. 4A), and the resulting viral fraction “Fr. B” was pooled, relying on the average retention volume that was observed above. Fr. B was then diluted 6.7-fold with 0.9% NaCl, loaded onto a CHAp column, and eluted with a linear concentration gradient of NaPB (pH 7.2) with 1M NaCl (FIG. 4B). The peak TCID₅₀ was detected at 3 to 7 mL (Fr. C) of the retention volume and contained highly purified capsid proteins (FIGS. 4B and 4C).

The contents of the experiments in FIG. 4 will be described below.

FIG. 4 shows the sequential two-step purification of Sabin type 2 virus.

(A) Purification of Sabin type 2 virus by pH gradient elution on a ceramic fluoroapatite (CFAp) column (Step 1).

Column, CFAp;

sample (volume), cell culture supernatant containing Sabin type 2 virus (10 mL);

wash, 10 mM sodium phosphate buffer (NaPB) (pH 6.4, 9 mL) and 300 mM NaPB (pH 6.4, 20 mL);

equilibration, 300 mM NaPB (pH 5, 15 mL);

elution, linear pH gradient from pH 5 to pH 8.2 at 300 mM NaPB for 10 mL; and

wash after separation, 300 mM NaPB (pH 8.2, 5 mL) and 600 mM NaPB (pH 8.2, 10 mL).

Lines are the same as in FIG. 2.

Fr. B was pooled and further purified in Step 2.

(B) Removal of double-stranded DNA (dsDNA) from the Fr. B fraction by NaPB elution with 1M NaCl on a ceramic hydroxyapatite (CHAp) column (Step 2)

Column, CHAp;

sample (volume), Fr. B obtained in (A) diluted 6.7-fold with 0.9% NaCl (17 mL);

buffer, pH 7.2;

column wash, 10 mM NaPB (13 mL);

equilibration, 10 mM NaPB (14 mL) with 1 M NaCl;

elution, linear concentration gradient from 10 mM to 187 mM NaPB with 1 M NaCl for 10 mL; and

wash, 600 mM NaPB (10 mL).

Fr. C was pooled for further evaluation.

(C) Sodium dodecyl sulfate-polyacrylamide gel electrophoresis (SDS-PAGE) analysis of the pooled fraction obtained through the two-step purification process

The pooled Fr. C fraction obtained in (B) and the cell culture supernatant were concentrated 100- and 10-fold, respectively, by ultrafiltration using a molecular weight cutoff of 10,000. The sizes are given in kDa on the left. Arrows indicate the major proteins in Fr. C.

TCID₅₀, median tissue culture infectious dose.

### Discussion

When manufacturing vaccines for use in clinical trials, preclinical studies, or commercial medicines, the aim is to minimize impurities and maximize the recovery of the vaccine during purification. The impurities are typically DNA and proteins derived from host cells, components of the cell culture medium, and/or some ligands released from purification process. In this study, we successfully purified Sabin type 2 virus directly from the cell culture supernatant without using any concentrating or buffer exchange processes, achieving the mean recovery rate in TCID₅₀ of 58.7%±30.0%, and the mean removal rates of proteins and double-stranded DNA of 99.95±0.006% and 99.99%±0.003%, respectively, (three independent experiments).

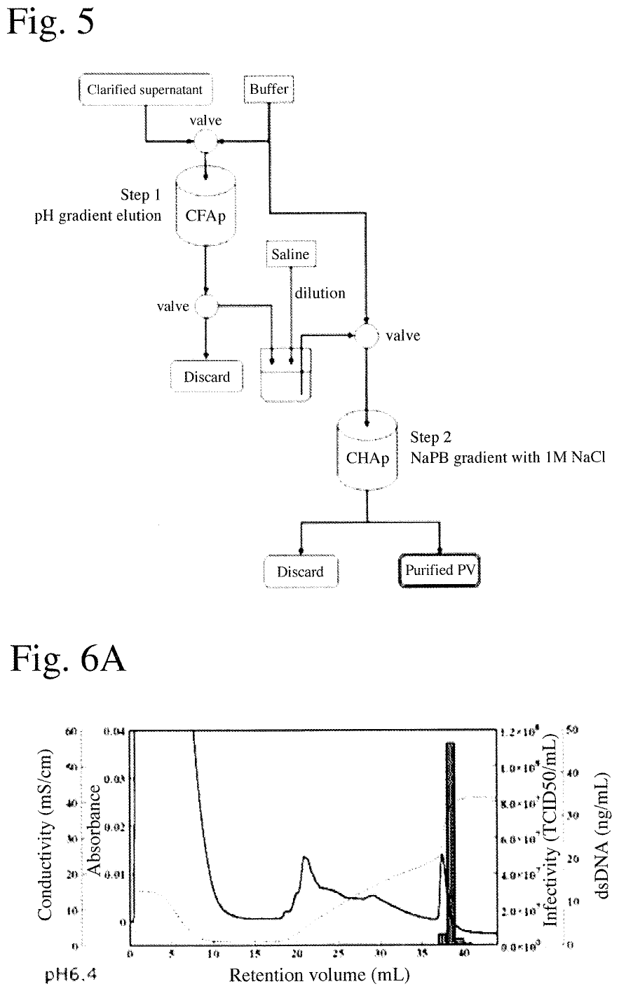

In Step 1, the cell culture supernatant was loaded directly onto a CFAp column and eluted with a pH gradient. In Step 2, the resulting virus fraction was diluted with 0.9% NaCl, loaded on a CHAp column, and eluted with an NaPB gradient in the presence of a high concentration of NaCl. Because both Steps 1 and 2 were highly reproducible, the viral fraction could be obtained without the need for any detection processes. This retention-volume-dependent fractionation should reduce the time and cost required to make vaccines. Furthermore, although the procedure currently contains multistep and offline purification processes, it should be possible to automate all the purification steps in the future. This two-step chromatographic purification is also easier to scale up than other current procedures because it was constructed using fully scalable processes (FIG. 5).

Abbreviations in FIG. 5 will be described below:

FIG. 5 shows a Tandem column system.

CFAp, ceramic fluoroapatite;

CHAp, ceramic hydroxyapatite;

NaPB, sodium phosphate buffer; and

PV, poliovirus.

Two-step chromatographic purification may also be useful for purifying other poliovirus strains such as Salk and Sabin types 1 and 3, as well as other nonenveloped viruses including hepatitis A virus, norovirus and feline calicivirus. However, this process does have some methodological limitations because Step 1 uses a low pH that will disrupt some viruses. Consequently, it may not be applicable for the purification of flaviviruses, which are unstable at acidic pH values. In addition to their role in vaccine production, infective virus purification techniques also play an important role in the field of gene therapy. In particular, there is an urgent clinical need for the large-scale purification of adenovirus because this virus is considered one of the most suitable platforms for gene transduction. Several other gene therapies have also yielded promising results for the use of virus vectors that express clinically relevant genes. For example, oncolytic adenovirus has been drawing attention recently as an antitumor medicine, and dozens of studies using this virus are now registered in Clinical-Trials.Gov (https://clinicaltrials.gov/), a database of clinical studies conducted around the world. This new purification method should be appropriate for adenoviruses that are resistant to acidic purification conditions.

Supporting Information

FIGS. 6A to 6C

Chromatograms of Sabin type 2 virus-containing cell culture supernatant separated on a ceramic hydroxyapatite (CHAp) column at different pH values

Separation occurred at buffer pH values of (A) 6.4, (B) 7.2, and (C) 8.2.

Column, CHAp (40 μm);

sample (volume), cell culture supernatant containing Sabin type 2 virus (5 mL);

column wash and equilibration, 10 mM sodium phosphate buffer (NaPB; 11 mL);

elution, linear concentration gradient from 10 mM to 300 mM NaPB for 20 mL; and

wash after separation, 600 mM NaPB (8 mL).

blue line, ultraviolet (UV) absorbance at 280 nm;

black broken line, conductivity;

red line, infectivity in median tissue culture infectious dose (TCID₅₀); and

purple line, double-stranded DNA (dsDNA) contents.

FIGS. 7A to 7C

Chromatograms of dengue virus type 1-containing cell culture supernatant separated on a ceramic hydroxyapatite (CHAp) column at different pH values

Separation occurred at buffer pH values of (A) 6.4, (B) 7.2, and (C) 8.2.

Column, CHAp (40 μm);

sample (volume), cell culture supernatant containing dengue virus type 1 (10 mL);

column wash and equilibration, 10 mM sodium phosphate buffer (NaPB; 10 mL);

elution, linear concentration gradient from 10 mM to 300 mM NaPB for 30 mL and from 300 mM to 600 mM for 8 mL; and

wash after separation, 600 mM NaPB (5 mL).

Lines are the same as in FIGS. 6A to 6C except for the red line, which indicates virus activity in the hemagglutination (HA) assay.

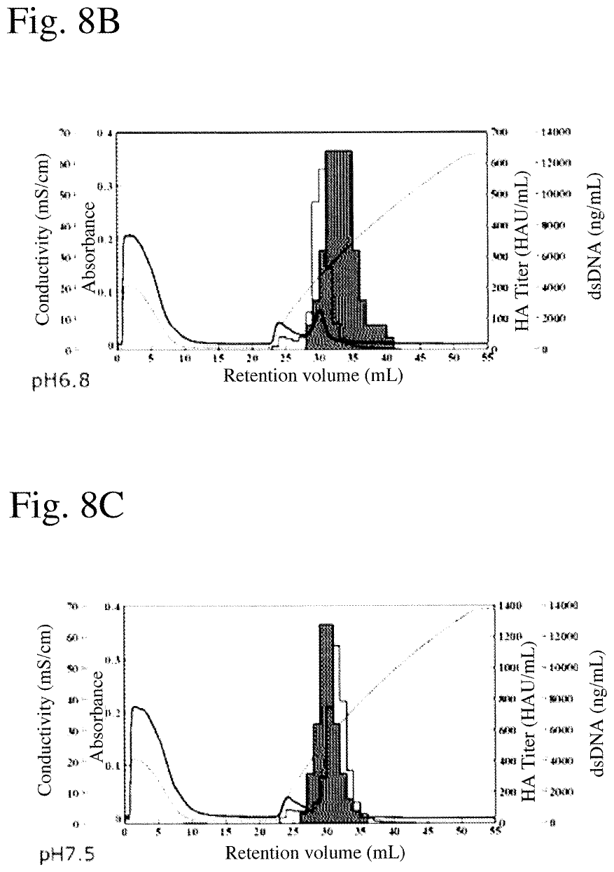

FIGS. 8A to 8C

Chromatograms of influenza virus NYMC X-181 containing cell culture supernatant separated on a ceramic hydroxyapatite (CHAp) column at different pH values

Separation occurred at buffer pH values of (A) 6.5, (B) 6.8, and (C) 7.5.

Column, CHAp (40 μm);

sample (volume), cell culture supernatant containing influenza virus NYMC X-181 (5 mL);

column wash and equilibration, 10 mM sodium phosphate buffer (NaPB; 15 mL);

elution, linear concentration gradient from 10 mM to 600 mM NaPB for 30 mL; and

wash, 600 mM NaPB (5 mL).

Lines are the same as in FIGS. 6A to 6C except for the red line, which indicates virus activity in the hemagglutination (HA) assay.

FIGS. 9A to 9D

Chromatograms of influenza virus NYMC X-181 containing cell culture supernatant in the presence of NaCl

The buffer contained (A) 0 M, (B) 0.14 M, (C) 0.5 M, and (D) 1 M NaCl.

Column, ceramic hydroxyapatite (CHAp);

sample (volume), cell culture supernatant containing influenza virus NYMC X-181 (5 mL);

buffer pH, 7.5;

column wash and equilibration, 5 mM sodium phosphate buffer (NaPB) with NaCl (15 mL);

elution, linear concentration gradient from 5 mM to 600 mM NaPB with NaCl for 30 mL; and

wash after separation, 600 mM NaPB (5 mL).

Lines are the same as in FIGS. 6A to 6C except for the red line, which indicates virus activity in the hemagglutination (HA) assay.

1. A purification method for removing a contaminant from a composition containing a virus particle and the contaminant, the purification method comprising:
a preparation step of preparing the composition containing a virus particle and a contaminant;
a first adsorption step of adsorbing the virus particle on a first adsorbent composed of a calcium phosphate compound by contacting the composition with the first adsorbent;
a first elution step of eluting the virus particle from the first adsorbent to obtain a first eluate;
a second adsorption step of adsorbing the virus particle on a second adsorbent composed of a calcium phosphate compound by contacting the first eluate with the second adsorbent; and
a second elution step of eluting the virus particle from the second adsorbent to obtain a second eluate;
one of the first elution step and the second elution step comprising pH gradient elution, and the other comprising salt concentration gradient elution.

2. The purification method according to claim 1, wherein the pH gradient elution comprises a step of changing a pH value of a solution supplied to the first adsorbent from a first pH value to a second pH value,
the first pH value is acidic, and
the second pH value is neutral or alkaline.

3. The purification method according to claim 2, wherein the first pH value is 6.0 or lower, and the second pH value is 7.5 or higher.

4. The purification method according to claim 1, wherein the salt concentration gradient elution comprises a step of changing a salt concentration of a solution supplied to the second adsorbent from a first concentration to a second concentration,
the first concentration is 10 mM or less, and
the second concentration is 100 mM or more.

5. The purification method according to claim 1, wherein the first elution step comprises the pH gradient elution, the first adsorbent comprising fluoroapatite, and
the second elution step comprises the salt concentration gradient elution, the second adsorbent comprising hydroxyapatite.

6. The purification method according to claim 5, wherein the first elution step is conducted before the second elution step.

7. The purification method according to claim 1, wherein the virus particle is a poliovirus particle.

8. A method for producing a virus particle, the method comprising steps of preparing a composition containing a virus particle and a contaminant, and removing the contaminant from the composition by the purification method recited in claim 1.

9. The purification method according to claim 2, wherein the virus particle is a poliovirus particle.

10. A method for producing a virus particle, the method comprising steps of preparing a composition containing a virus particle and a contaminant, and removing the contaminant from the composition by the purification method recited in claim 2.
